(19) United States
(12) Patent Application Publication
(10) Pub. No.: US 2017/0320965 A1
(43) Pub. Date: Nov. 9, 2017

(54) PAN-REACTIVE ANTIBODIES TO DUOCARMYCINS

(71) Applicant: SYNTHON BIOPHARMACEUTICALS B.V., Nijmegen (NL)

(72) Inventors: David EGGING, Nijmegen (NL); Patrick Henry BEUSKER, Nijmegen (NL); Ellen MATTAAR, Nijmegen (NL); Ebo BOS, Nijmegen (NL)

(73) Assignee: SYNTHON BIOPHARMACEUTICALS B.V., Nijmegen (NL)

(21) Appl. No.: 15/512,934

(22) PCT Filed: Sep. 22, 2015
(86) PCT No.: PCT/EP2015/071671

§ 371 Date: Mar. 21, 2017

(30) Foreign Application Priority Data
Sep. 22, 2014 (EP) 14185773.0

(51) Int. Cl. C07K 16/44; G01N 33/543

(52) U.S. Cl. CPC C07K 16/44; G01N 33/54306; C07K 2317/565

(57) ABSTRACT

The present invention relates to a monoclonal, pan-reactive antibody to duocarmycins. The monoclonal, pan-reactive antibody of the present invention may be used to detect, isolate and/or quantify a duocarmycin-containing antibody-drug conjugate in a biological or non biological sample, for example using an immunoassay.

### FIELD OF THE INVENTION

The present invention relates to monoclonal, pan-reactive antibodies to duocarmycins. The pan-reactive antibodies of the present invention may be used to detect, purify, isolate and/or quantify duocarmycin-containing antibody-drug conjugates in biological and non-biological samples, for example using an immunoassay.

### BACKGROUND OF THE INVENTION

Antibody-drug conjugates (ADCs) are an emerging new class of targeted therapeutics having an improved therapeutic index over traditional therapy. ADCs are complex molecules composed of a biologically active cytotoxic (anticancer) small-molecule payload or drug linked to an antibody via a chemical linker.

Adequate control of the quality of these biotherapeutic molecules and assessing their pharmacokinetic (PK) characteristics are crucial for batch-to-batch reproducibility of therapeutic efficacy, proper dosing and patient safety.

As is known from the art, monoclonal antibodies (mAbs) can be used in methods for the detection, quantification and purification and/or isolation of the specific antigen or substances containing the cognate epitope, e.g. in an immunoassay. These monoclonal antibodies may either be directed against the antibody, and/or a linking moiety and/or against the cytotoxic drug(s), often a small molecule toxin, or an epitope of any of the aforementioned.

It is known in the art that antibodies can be obtained by immunizing animals such as mice by exposure to immunogenic material. Unlike proteins such as antibodies, small molecules (haptens) are not immunogenic by themselves and must be conjugated to a suitable immunogenic carrier, thus forming an immunogen, when attempting to generate antibodies against the hapten.

For example, WO 94/24304A discloses immunizing mice for generating monoclonal antibodies to the small molecule rapamycin and to a 40-O-alkylated derivative thereof and their use in an enzyme-linked immunosorbent assay (ELISA) for monitoring blood levels of rapamycin in patients.

US 2006/0073528A discloses a method for quantifying an unconjugated anti-PSMA antibody in a sample comprising cytotoxic maytansinoid DM1 conjugated to an anti-PSMA antibody. The method requires developing three separate antibodies, i.e., one for binding the immunoconjugate, one for removing unconjugated DM1 and one for removing unconjugated anti-PSMA antibody. US 2006/0073528A further teaches that for each different maytansinoid in the ADC an antibody specific for that particular maytansinoid should be developed to deplete the ADC, making the method even more laborious.

U.S. Pat. No. 5,662,911 discloses that multiple benzodiazepine variants may be detected in one assay when using sera containing polyclonal antibodies obtained from mice. However, such sera cannot be manufactured in a reproducible manner.

There is thus a need for an assay which can be used for a large number of different, but structurally-related cytotoxic drugs, e.g., duocarmycins, conjugated to a targeting moiety, such as an antibody.

### BRIEF DESCRIPTION OF THE INVENTION

The present invention relates to monoclonal, pan-reactive antibodies to duocarmycins. The pan-reactive antibodies of the present invention may be used to detect, purify, isolate and/or quantify duocarmycin-containing antibody-drug conjugates in biological and non-biological samples, for example using an immunoassay.

In a first aspect, the present invention relates to a monoclonal antibody that specifically binds antibody-drug conjugates containing a DNA alkylating moiety of formula (I)

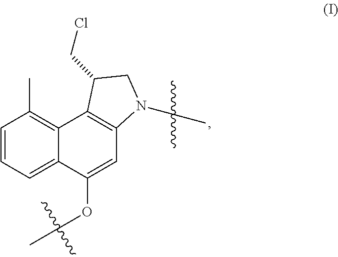 [SMILES: *Oc1cc2c(c3c(C)cccc13)[C@H](CCl)CN2*]

wherein said antibody specifically binds at least two different antibody-drug conjugates that contain said DNA alkylating moiety.

Other aspects of the present invention include a process for making the monoclonal antibodies and the use of said monoclonal antibodies in an immunoassay.

### BRIEF DESCRIPTION OF THE DRAWINGS

FIG. 1. BIA results of selected monoclonal, pan-reactive anti-duocarmycin antibodies.

FIG. 2. Structures of duocarmycin containing antibody-drug conjugates.

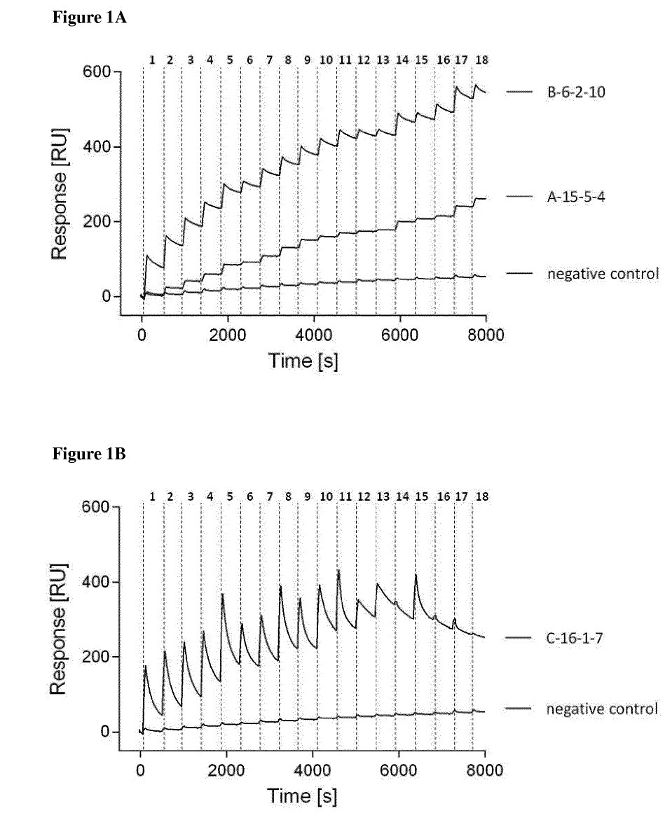

### DETAILED DESCRIPTION OF THE PRESENT INVENTION

Antibody-drug conjugates (ADCs) are emerging as a new class of therapeutics that combines the efficacy of small-molecule therapeutics with the targeting ability of antibodies. By combining these two components into a single new molecular entity, cytotoxic small molecule drugs can be delivered to target tissues, thereby enhancing efficacy while reducing the potential systemic toxic side effects of the small molecule.

Antibodies have been conjugated to a variety of cytotoxic drugs, including small molecules that bind DNA (e.g. anthracyclines), alkylate or crosslink DNA (e.g. duocarmycins or pyrrolobenzodiazepine dimers, respectively), cause DNA strand breaks (e.g. calicheamicins) or disrupt microtubules (e.g. maytansinoids and auristatins).

Duocarmycins, a class of structurally-related toxins first isolated from a culture broth of Streptomyces species, are members of a family of antitumor antibiotics that include duocarmycin A, duocarmycin SA, and CC-1065. These extremely potent agents appear to derive their biological activity from an ability to sequence-selectively alkylate DNA at the N3 position of adenine in the minor groove, which initiates a cascade of events that terminates in an apoptotic cell death mechanism.

The present invention relates to a monoclonal antibody that specifically binds antibody-drug conjugates containing a DNA alkylating moiety of formula (I)

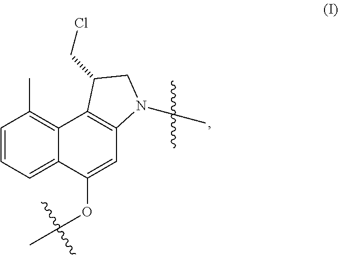 [SMILES: *Oc1cc2c(c3c(C)cccc13)[C@H](CCl)CN2*]

wherein said antibody specifically binds at least two different antibody-drug conjugates that contain said DNA alkylating moiety.

The N-atom in the above formula (I) is connected to a duocarmycin DNA binding moiety, whereas the O-atom is connected via a linker to an antibody.

Earlier work indicates that for each drug moiety in an ADC, drug-specific monoclonal antibodies must be generated. But when developing and testing a large number of different, but structurally-related ADCs, i.e., all containing the DNA alkylating moiety of a duocarmycin as in formula (I), but varying in antibody, linker and/or duocarmycin binding moiety, having to generate separate assays for each different ADC is laborious and expensive and thus undesirable.

Surprisingly, the present inventors found that it is was possible to develop one assay or method for detecting, purifying, quantifying or isolating multiple duocarmycin ADCs using one single monoclonal, pan-reactive antibody that can bind multiple duocarmycin ADCs.

They not only succeeded in raising antibodies against a common epitope in members of the class of duocarmycins, namely the DNA alkylating moiety of formula (I), but in addition found against expectation that this epitope is still accessible for the pan-reactive antibody when present in an ADC, irrespective of the nature of the antibody, linker and/or duocarmycin DNA binding moiety as present in the ADC. The monoclonal, pan-reactive antibodies of the present invention may be used for the detection, purification, quantification and isolation of duocarmycin-containing ADCs in biological and non-biological samples.

The term “pan-reactive antibody” as used throughout the present specification is defined as any antibody which selectively binds at least two members of a panel of structurally-related duocarmycin-containing antibody-drug conjugates, all containing the same DNA alkylating moiety. Preferably, at least three, four, five, six, seven, eight, or nine members are bound, more preferably at least ten, and even more preferably at least fifteen members of a panel of structurally-related duocarmycin-containing antibody-drug conjugates are bound.

The term “antibody” as used throughout the present specification is a monoclonal antibody (mAb) or an antigen binding fragment thereof, e.g. a Fab, Fab' or F(ab')₂ fragment, a single chain (sc) antibody, a scFv, a single domain (sd) antibody, a diabody, or a minibody. Antibodies may be of any isotype such as IgG, IgA or IgM antibodies. Preferably, the antibodies are an IgG, more preferably an IgG1 or IgG2.

The term “linker” as used in accordance with the present invention may be any non-cleavable or cleavable chemical linker as known in the art, for example succinimidyl 4-(N-maleimidomethyl)cyclohexane-1-carboxylate (SMMC) or the linkers as described in WO 2010/062171A and WO 2011/133039A.

Duocarmycin DNA binding moieties in the context of the present invention encompass the DNA binding units of formulae (I) and (II) as disclosed in WO 2009/017394A, and moieties DB1 to DB9 as disclosed in WO 2010/062171A and WO 2011/133039A.

In one embodiment, the monoclonal antibody of the present invention binds at least three, preferably at least four, five, six, seven, eight, or nine, more preferably at least ten, and even more preferably at least fifteen different antibody-drug conjugates that contain said DNA alkylating moiety.

In a second embodiment, the present invention relates to a monoclonal antibody binding to duocarmycin containing ADCs of formula (II)

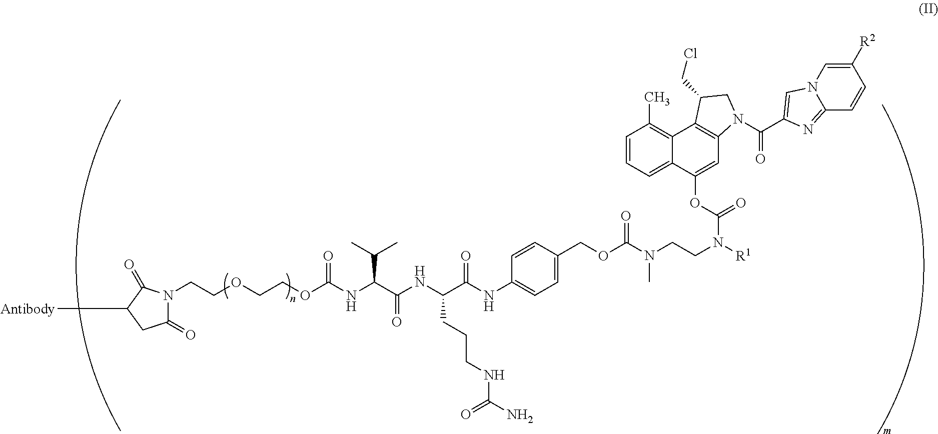

wherein

n is 0-3, preferably 0 or 1,

m is 1-6, preferably 1-4, and represents an average drug-to-antibody ratio (DAR) of from 1 to 6, preferably of from 1 to 4,

R¹ is selected from the group consisting of

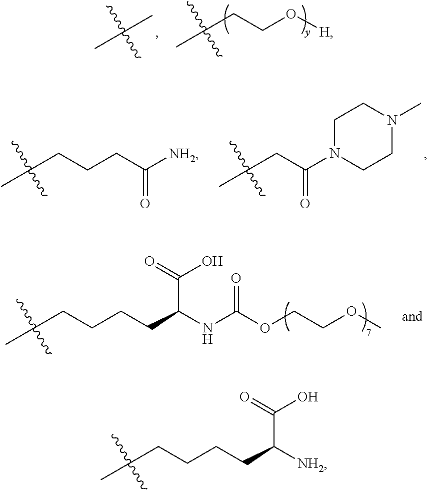

y is 1-16, and

R² is selected from the group consisting of

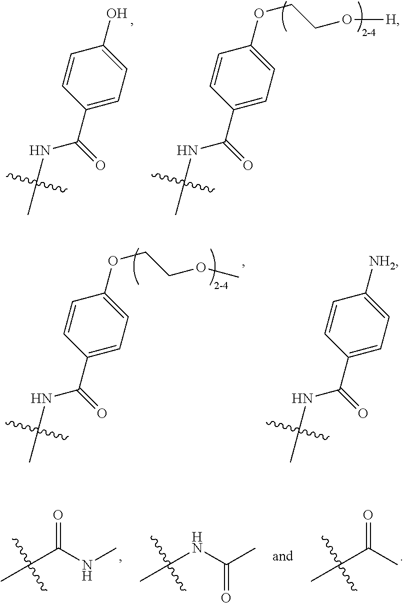

In structural formula (II), n represents an integer from 0 to 3, while m represents an average DAR of from 1 to 6. As is well-known in the art, the DAR and drug load distribution can be determined, for example, by using hydrophobic interaction chromatography (HIC) or reversed phase high-performance liquid chromatography (RP-HPLC). HIC is particularly suitable for determining the average DAR.

In one particular aspect of the above embodiment R² is selected from the group consisting of

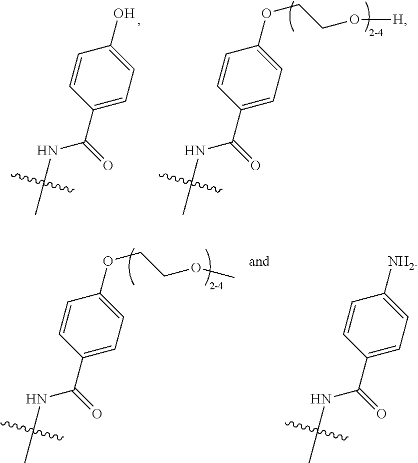

In a second particular aspect of the above embodiment the antibody is an anti-annexin A1 antibody, an anti-CD115 antibody, an anti-CD123 antibody, an anti-CLL-1 antibody, an anti-HER2 antibody, an anti-c-MET antibody, an anti-MUC1 antibody, an anti-PSMA antibody, an anti-5T4 antibody or an anti-TF antibody. Preferably, the antibody is anti-HER2 antibody trastuzumab.

In a third embodiment of the present invention, the monoclonal antibody is obtainable by raising antibodies against hapten A, B or C

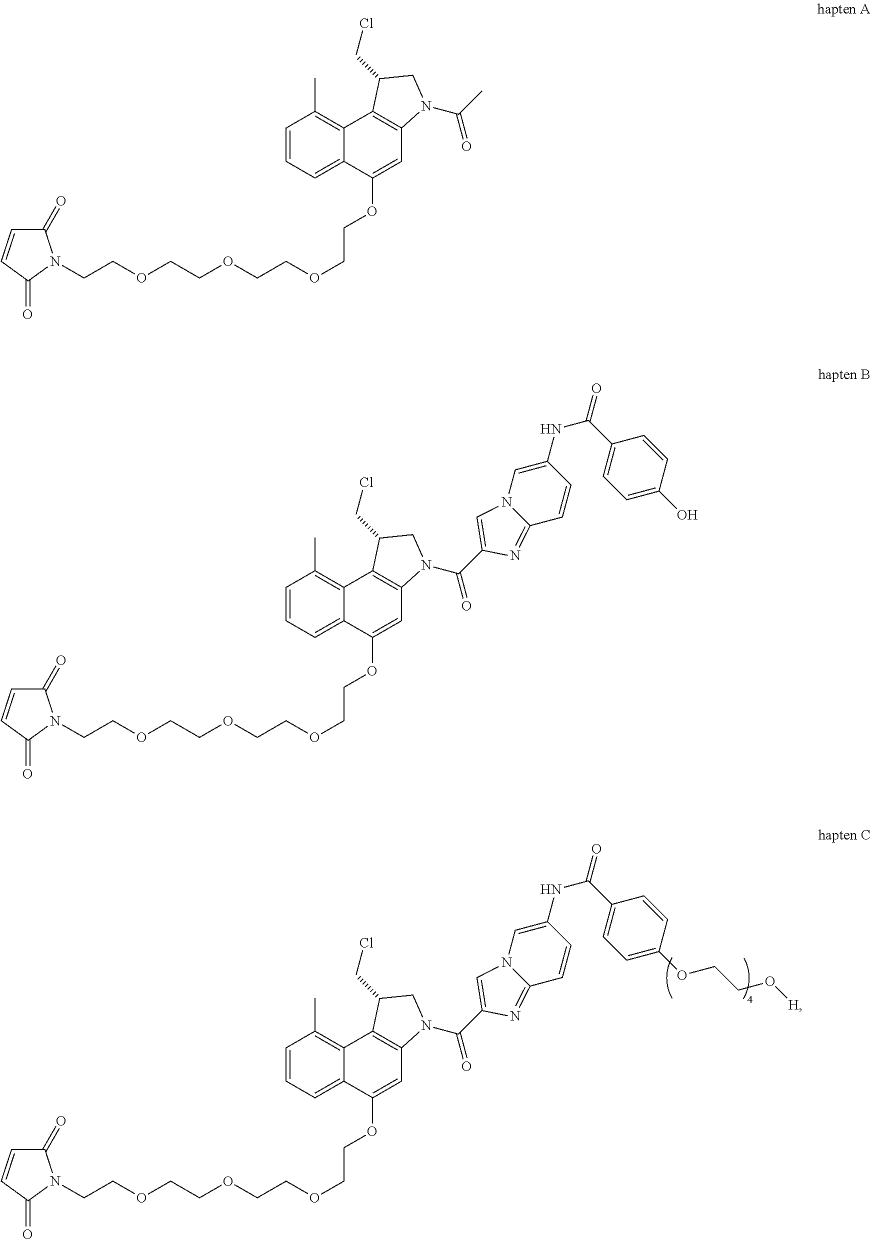

after conjugating the hapten to a carrier protein and immunizing mice with the hapten-carrier protein conjugate.

The term “carrier protein” as used throughout the present specification is defined as any protein to which the hapten may be attached to render the hapten-carrier protein conjugate immunogenic. Suitable carrier proteins are well known and may be readily chosen by the person skilled in the art. Particularly suitable carrier proteins include proteins such as keyhole limpet hemocyanin (KLH), bovine serum albumin (BSA), and ovalbumin (OVA).

In accordance with the present invention hybridoma cell lines (or hybridomas) have been created that produce monoclonal antibodies which bind duocarmycin-containing antibody-drug conjugates.

The present invention further relates to a process for making a monoclonal, pan-reactive antibody as described hereinabove comprising:
(i) conjugating hapten A, B, or C to a carrier protein,
(ii) immunizing mice with a hapten-carrier protein conjugate of step (i),
(iii) recovering splenocytes or lymphocytes from said immunized mice,
(iv) generating hybridomas,
(v) selecting stable, antibody-producing hybridomas,
(vi) contacting antibodies produced by hybridomas of step (v) with a panel of duocarmycin containing antibody drug conjugates, and
(vii) selecting antibodies that are reactive against at least two of the panel of duocarmycin-containing antibody-drug conjugates.

The preparation of hybridomas may be accomplished using conventional techniques such as those described by Kohler and Milstein in Nature, 256:495-497 (1975), Koprowski et al. in U.S. Pat. No. 4,196,265, Wands in U.S. Pat. No. 4,271,145, and Stanker et al. in U.S. Pat. No. 5,466,784. Briefly, these conventional techniques comprise the following steps: administering an immunogenic conjugate to a suitable animal species to effect an immunogenic challenge and recovering antibody-producing cells sensitized to said conjugate; immortalizing said antibody-producing cells by fusion with suitable myeloma cells; and recovering the monoclonal antibody from a selected immortalized cell line thus-established.

In accordance with the present invention, the process for the preparation and selection of suitable hybridomas comprises the steps of:

1) preparation of haptens,

2) preparation of corresponding immunogens,

3) immunization of immunologically competent animals with said immunogens,

4) analysis of titers,

5) recovering splenocytes or lymphocytes from said animals,

6) generation of hybridomas,

7) selection of hybridomas (first screening),

8) selection of monoclonal, pan-reactive antibodies (second screening), and

9) cloning of stable, antibody-producing (hybridoma) cell lines.

Ad 1) Preparation of Haptens

In accordance with one embodiment of the present invention three different haptens, i.e., haptens A, B and C, are prepared, all three sharing at least a common duocarmycin-derived DNA alkylating moiety, as depicted below.

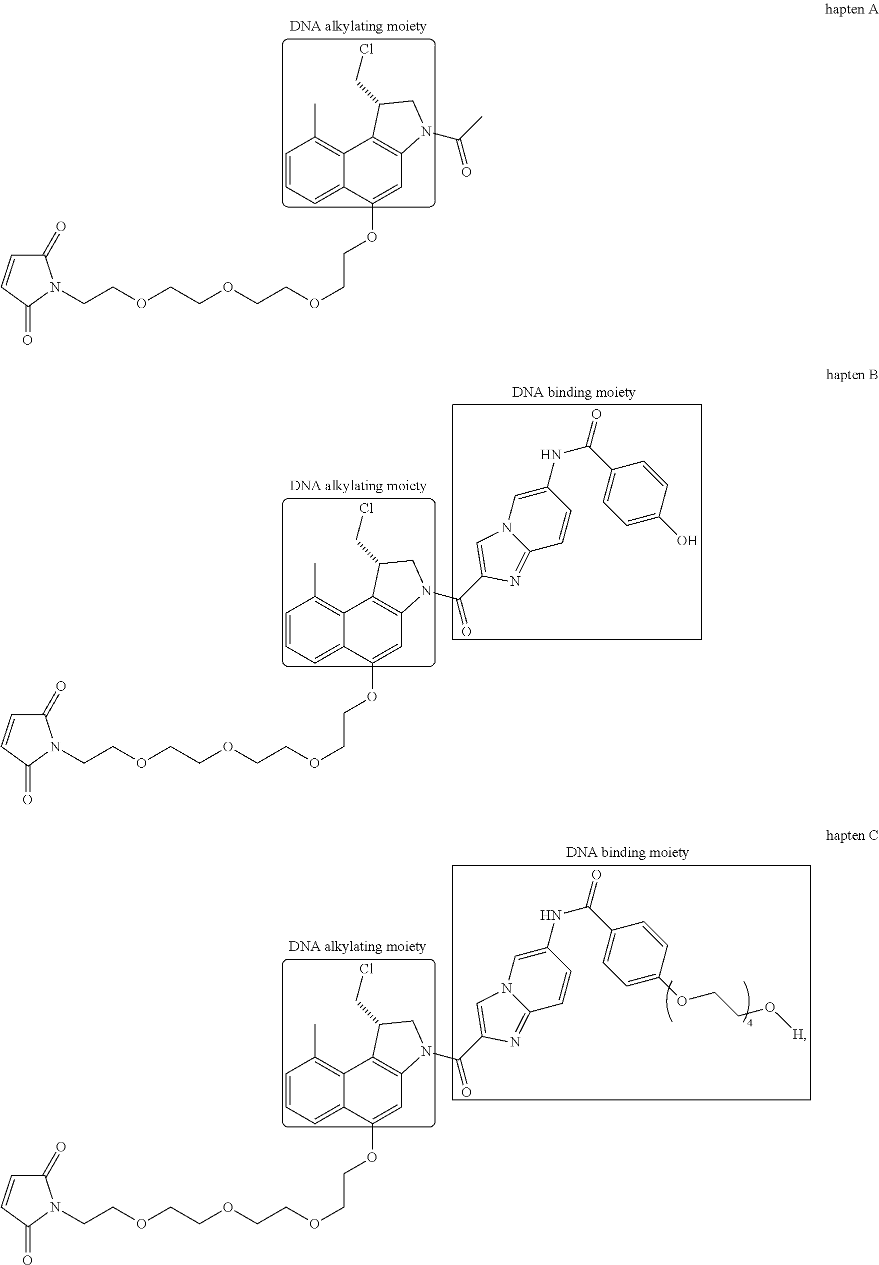

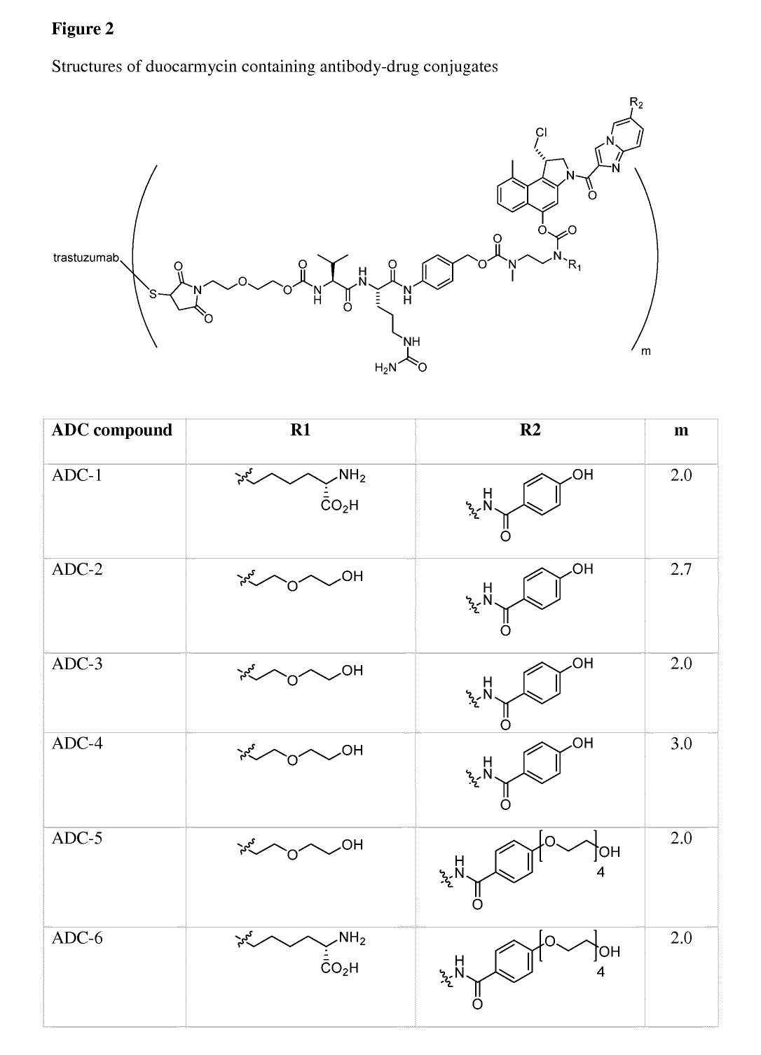

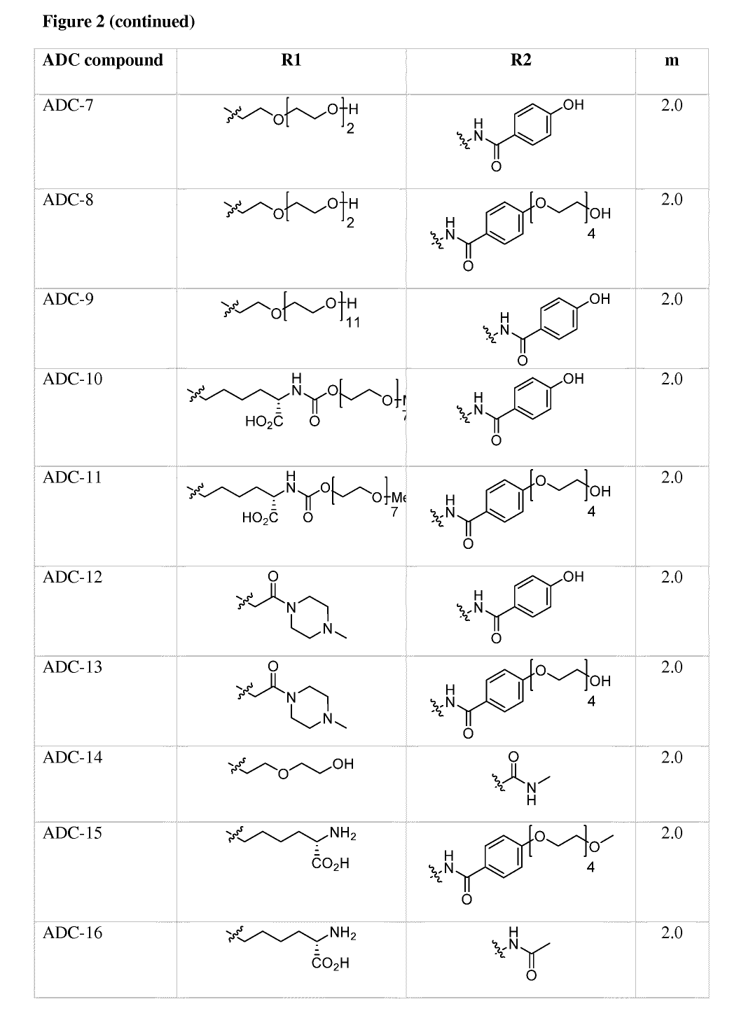

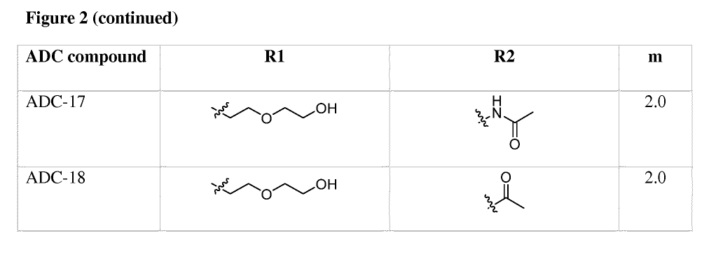

Hapten A essentially only contains the DNA alkylating moiety of a duocarmycin prodrug. Haptens B and C contain said same DNA alkylating moiety as well as a DNA binding moiety, which is structurally similar in haptens B and C. Haptens A, B and C are prepared from starting materials and following procedures well-known to the person skilled in the art, see WO 2009/017394A (e.g. see reaction scheme in FIG. 2), WO 2010/062171A (e.g. see reaction scheme in FIG. 2), and WO 2011/133039A (e.g. see compounds on pages 87 and 89).

Ad 2) Preparation of Corresponding Immunogens

The immunization agents of one embodiment of the present invention, i.e., haptens A, B and C, are linked to a suitable immunogenic carrier to form an immunogenic conjugate. The immunogenic carrier may be conjugated to the hapten directly or through an optional spacer.

In a specific embodiment of the present invention, the immunogenic conjugate is prepared by linking the hapten to SATA-modified BSA (N-succinimidyl-S-acetylthioacetate-modified bovine serum albumin).

Ad 3) Immunization of Immunologically Competent Animals with said Immunogens

To generate antibody-producing splenocytes or lymphocytes, an immunizing preparation comprising the hapten-carrier protein conjugate is injected into an immunologically competent animal. The preparation may also contain other proteins, although pure or partially pure compositions of the conjugate in a pharmaceutically acceptable carrier are preferred.

In a specific embodiment of the present invention the hapten A, B or C-carrier protein conjugate is injected into mice.

Inoculation of the animal can be done by various routes. In a specific embodiment of the present invention, the inoculation is done intraperitoneally in five mice over a period of 39 days with a composition of the conjugate in Freund's complete (priming) or incomplete adjuvant (boosting) eliciting a good antibody response.

The person skilled in the art will recognize that other routes of administration, immunization schedules, carriers or adjuvants may be used as well.

Ad 4) Analysis of Titers

Titers are measured in blood samples from an immunologically competent animal using an immunoassay to determine the presence of antibodies reactive to the antigen in the animal's blood serum. Suitable immunoassays are well known and may be readily chosen by the person skilled in the art. Without being limited thereto, a preferred immunoassay is an ELISA assay in which the solid phase, such as beads or the wells of a microtiter plate are provided with a capture reagent. In a specific embodiment of the present invention, the ELISA is performed using hapten A, B or C conjugated to SATA-modified OVA (ovalbumin) for the determination of the presence of mouse IgG in antisera.

The detection is performed using methods known to the person skilled in the art. In a specific embodiment of the present invention, the detection is performed using an anti-mouse enzyme conjugate (to monitor antibody formation), in particular goat anti-mouse IgG conjugated to alkaline phosphatase.

Ad 5) Recovering Splenocytes or Lymphocytes from said Animals

Immunologically competent animals with sufficiently high titers are sacrificed and spleens are dissected. In a specific embodiment of the present invention, mice are sacrificed by inhalational isoflurane anesthesia followed by cervical dislocation. Spleens are aseptically removed, pooled and homogenized.

Ad 6) Generation of Hybridomas

Splenocytes or lymphocytes recovered from immunized animals are fused with continuously replicating tumor cells, and cultured to produce hybridoma cells. In a specific embodiment of the present invention, spleen cells are immortalized by cell fusion using myeloma cell line SP2/0-Ag14.

Fusion and culture of cells can be performed using conventional techniques. In a specific embodiment of the present invention, in accordance with one well-known effective procedure, the splenocytes and tumor cells are fused by exposure to polyethylene glycol. Hybrid cells are selected by culture in hypoxanthine-aminopterin-thymidine (HAT) medium, whereby nonfused tumor cells are killed by HAT and splenocytes die out, leaving only the hybrid cells. The resultant hybridomas are then grown in suitable culture medium and assayed for antibody production.

Ad 7) Selection of Hybridomas (First Screening)

The samples of the supernatant culture fluid comprising the resultant hybrid cells are screened with a suitable assay for the production of antibodies reactive to the antigen. Suitable immunoassays are well known and may be readily determined by the person skilled in the art. Without being limited thereto, a preferred immunoassay is the ELISA assay in which the solid phase such as beads or the wells of a microtiter plate are coated with a capture reagent. In a specific embodiment of the invention the ELISA is performed using hapten A, B or C conjugated to SATA-modified OVA for the detection of mouse IgG in hybridoma cell culture supernatants. The detection is performed with methods known to the person skilled in the art. In a specific embodiment of the present invention, the detection is performed using an anti-mouse enzyme conjugate (to monitor antibody formation), in particular goat anti-mouse IgG conjugated to alkaline phosphatase.

In a specific embodiment of the present invention, all cultures that were found to produce e.g. hapten A-OVA-reactive antibodies in the primary screening, are cultured for a few days and then re-tested to analyze their antibody reactivity against e.g. hapten A-OVA. The re-screening allows for the selection of hybridomas with a documented antibody reactivity profile as well as cultures with stable antibody secretion.

Ad 8) Selection of Monoclonal, Pan-Reactive Antibodies (Second Screening)

The samples of the supernatant culture fluid are screened with a suitable assay for the selection of pan-reactive antibodies reactive to the antigen. Suitable immunoassays are well known and may be readily determined by the person skilled in the art. A preferred immuno-assay is the Biomolecular Interaction Analysis (BIA) assay in which the sensor chip surface is modified by immobilization of polyclonal anti-mouse which results in a surface that can bind any mouse monoclonal antibodies.

In a specific embodiment of the present invention, the BIA assay is performed using an anti-hapten A, B or C monoclonal antibody captured on a polyclonal anti-mouse surface to a high binding level (capture reagent). Subsequently, a panel of duocarmycin-containing antibody-drug conjugates is injected sequentially and the signal (RU, resonance or responsive units) indicating binding is recorded. The panel of duocarmycin derivatives contain the same DNA alkylating moiety as a common structural moiety (as shown hereinabove), but have a different DNA binding moiety, and/or linker, and/or antibody. Monoclonal, pan-reactive antibodies are selected based on surface plasmon resonance (SPR), which response is directly proportional to the mass increase on the surface. An increase in mass on the surface is a positive binding reaction. Those antibodies binding to at least 3, preferably at least 5, more preferably at least 10, even more preferably at least 15, most preferably at least 18 different duocarmycin-containing antibody-drug conjugates having the same DNA alkylating moiety described hereinabove, are selected as pan-reactive antibodies in accordance with the present invention.

Ad 9) Cloning of Stable, Antibody-Producing (Hybridoma) Cell Lines

Cloning of hybridomas which are positive for pan-reactive antibody production is carried out by using methods known in the art. Hybridomas having a positive response in the assay screen are preferably expanded and subcloned one or more times by limiting dilution to assure monoclonality.

In a specific embodiment of the present invention, the cloning is performed as soon as possible using the limiting dilution method and after 6-8 days of cell culture, all plates are inspected to identify wells containing single cell colonies. The identified wells are fed with fresh medium, cultured and the best clones which continue to produce antibodies against hapten A, B or C are identified and re-cloned.

Three different monoclonal, pan-reactive antibodies (i.e., A-15-5-4, B-6-2-10 and C-16-1-7) show binding to a panel of 18 different duocarmycin-containing antibody-drug conjugates having a common DNA alkylating moiety as shown hereinabove. The structures of the 18 ADCs are depicted in FIG. 2.

Antibody Characterization

The antibodies which are prepared in accordance with the process of the present invention are characterized (e.g. isotype, molecular weight, isoelectric point) using conventional techniques known to a person skilled in the art. For example, the heavy and light chain isotypes of antibodies are determined using a mouse monoclonal antibody isotyping test kit from Serotec.

Antibody Application

The pan-reactive antibodies of the present invention may advantageously be used in a number of assays. One such assay is using a pan-reactive antibody in accordance with the present invention in a method for detecting, isolating or quantifying a duocarmycin-containing antibody-drug conjugate in a biological or non-biological sample comprising:

(i) subjecting said sample to an immunoassay using a monoclonal, pan-reactive antibody as described hereinabove, and

(ii) detecting, isolating or quantifying the resulting immune complex. Said assays typically rely on two common techniques, i.e., Enzyme Linked Immuno Sorbent Assay (ELISA) and Biomolecular Interaction Analysis (BIA), e.g. using a Biacore T200 instrument.

Typically, such immunoassay includes the steps of
(i) providing a solid phase having said monoclonal, pan-reactive antibody bound thereto,
(ii) contacting said solid phase with said biological or non-biological sample,
(iii) washing said support,
(iv) detecting a duocarmycin-containing antibody-drug conjugate attached to said support, and
(v) determining the quantity of said antibody-drug conjugate in said sample.

In one embodiment of the present invention, the pan-reactive antibodies of the present invention advantageously are used in PK assays which are necessary for carrying out animal and human preclinical and clinical studies. A capture antibody is immobilized on the surface of a microtiter plate. Subsequently, the antigen (analyte) is incubated in the well for a specific time. Detection occurs by binding of a second antibody labeled with an enzyme. Usually, incubation with an excess of an inert protein is performed to prevent non-specific binding to the surface. Non-reactive components are washed away. Specifically, a pan-reactive antibody in accordance with the present invention is coated to a 96 well Greiner High binding plate and is subsequently incubated with a sample containing a duocarmycin-containing linker drug or antibody-drug conjugate. Detection occurs by binding of biotinylated anti-idiotype Fab antibody, e.g. trastuzumab Fab. After incubation with streptavidin-HRP and an incubation with the enzyme-substrate solution containing TMB, the enzyme reaction is stopped and a signal is measured at A450 nm. Alternatively, the anti-idiotype Fab antibody may be coated to the microtiter plate, while using a biotinylated pan-reactive anti-hapten antibody and streptavidin-HRP for detection.

In a further embodiment of the present invention, a pan-reactive antibody in accordance with the present invention is used in a test, such as a CMC identity test, to characterize a (therapeutic) compound. A pan-reactive anti-hapten antibody immobilized (ligand) on a sensor chip or tip surface by (amine) coupling it at a certain level of resonance units (RU), and a series of concentrations of duocarmycin-containing antibody-drug conjugates are injected and binding characteristics are determined using procedures and methods that are well-known to the person skilled in the art. Alternatively, duocarmycin-containing antibody-drug conjugates are immobilized (ligands) on a sensor chip or tip surface by coupling at a certain level of resonance units (RU), and a concentration series of monoclonal, pan-reactive anti-hapten antibodies in accordance with the present invention are injected and binding characteristics are determined. Alternatively, instead of using a sensor chip or tip surface, said (CMC identity) test or other ((bio-)analytical, bioanalysis) test can also be carried out using other techniques, e.g. AlphaLISA, electrochemiluminescence (ECL), a radioimmunoassay (RIA), or a Western blotting membrane (after separation by SDS PAGE and transfer from the gel) using procedures and methods that are well-known to a person skilled in the art.

The present invention further relates to a kit for the detection or quantification of a duocarmycin-containing antibody-drug conjugate in a biological or non-biological sample comprising a monoclonal, pan-reactive antibody as described hereinabove and instructions for use. In the kit, the monoclonal, pan-reactive antibody of the invention can be present in a lyophilized form, as a frozen solution, or coated onto the surface of a microtiter plate.

The present invention still further relates to the use of a monoclonal, pan-reactive antibody as described hereinabove for the detection, purification, isolation and/or quantification of a duocarmycin-containing antibody-drug conjugate in a biological or non-biological sample or as a control sample and or reference.

### EXAMPLES

Preparation of Immunogens

The (lyophilized) maleimide-containing haptens A, B and C (11.16 mg hapten A, 10.45 mg hapten B, and 10.40 mg hapten C) were each conjugated to SATA-modified BSA.

BSA was dissolved at a concentration of 5 mg/mL in PBS, pH 7.5, containing 1 mM EDTA. 10 μL of the SATA solution (13.6 mg/mL in DMSO) was added to each mL of BSA solution. After incubation for 30 min at room temperature (RT), the modified protein was separated from unreacted SATA and reaction by-products by dialysis against PBS, pH 7.5, containing 1 mM EDTA. SATA-modified carrier protein was deacetylated by addition of 100 μL of 0.5 M hydroxylamine hydrochloride in PBS, pH 7.5, containing 25 mM EDTA per mL of the SATA-modified protein solution. The reaction mixture was stirred for 2 h at RT and finally dialyzed against PBS, pH 7.5, containing 1 mM EDTA.

150 μL of the maleimide-containing hapten A, B or C solution (10 mg/mL, in N-methyl-2-pyrrolidone, NMP) were added to the deacetylated BSA-SATA solution and incubated at RT for 1 h. The three reaction mixtures were dialyzed against PBS, pH 6.7. The deacetylated protein and the conjugates were tested in order to determine the degree of −SH modification using Ellman's reagent. The absorption of the activated protein and conjugates was measured at 412 nm. The total number of sulfhydryl groups per BSA molecule after the thiolation was found to be 48.

Immunization of Animals

Five mice per BSA-hapten antigen were immunized intraperitoneally over a period of 39 days with a water-in-oil emulsion that was prepared by emulsifying the antigen in equal volumes of Freund's complete adjuvant (priming) or incomplete adjuvant (boosting). The presence of antibodies reactive to each antigen in the animals' blood serum was confirmed by direct ELISA as described below.

Analysis of the Titers

Mice were bled throughout the immunization period in order to monitor the development of the immune response. A terminal bleed was taken after splenectomy and the serum was used as a positive control in the hybridoma screening ELISA. For serum preparation, the blood was initially incubated for 1 h at RT and then overnight at 4° C. The clot was pelleted by centrifugation at 12,000×g for 10 min and the supernatant serum was recovered and stored at −20° C.

The (lyophilized) maleimide-containing haptens A, B and C (11.16 mg hapten A, 10.45 mg hapten B and 10.40 mg hapten C) were each conjugated to SATA-modified OVA and used in an ELISA. The conjugation was performed by the same method as described above for the conjugation to SATA-modified BSA. The total number of sulfhydryl groups per ovalbumin molecule after the thiolation was approximately 22.

An ELISA was developed for the detection of mouse IgG in antisera (detection with an anti-mouse AP conjugate). All tests were performed in 96-well, flat-bottom polystyrene microwell plates (PAA, Cat# PAA38096X).

The wells were coated with 50 μL/well of antigen, i.e., hapten-SATA-modified OVA conjugate A, B or C, at 4 μg/mL in 0.1 M carbonate/ bicarbonate buffer, pH 9.6. After overnight incubation at 4° C., the wells were washed three times with washing buffer (Tris-buffered saline, TBS; 10 mM Tris, 200 mM NaCl, pH 7.8 containing 0.01% Triton X-100) and subsequently blocked by incubation with 200 μL/well of blocking buffer (2% FCS in TBS) for 1 h at RT and plate shaking. After another washing step, 100 μL/well of neat CSN (culture supernatant) or antiserum diluted into blocking buffer were incubated in the wells for 1 h at RT. CSN of SP2/0-Ag14 myeloma cells and a 1:100 dilution of the serum pool of mice used for hybridoma production were tested in parallel as negative and positive assay controls, respectively.

After washing the wells four times with washing buffer, bound mouse IgG antibodies were labeled with goat anti-mouse IgG conjugated to alkaline phosphatase (50 μL/well, 1:5000 in blocking buffer) by incubation for 1 h at RT and plate shaking. Plates were washed again and the alkaline phosphatase activity was measured after incubation in substrate buffer (2 mM 4-nitrophenylphosphate in 5% diethanolamine, 1 mM MgCl₂, pH 9.8) by reading the optical density at 405 nm (OD405 nm) using a Dynex Opsys MR microplate reader. Wells with OD405 nm values twofold higher than the average plate OD405 nm were considered as positive wells.

Recovering Splenocytes or Lymphocytes from said Animals

Mice were sacrificed by inhalational isoflurane anesthesia followed by cervical dislocation. Spleens were aseptically removed, pooled and homogenized.

Generation of Hybridomas

The spleen cells and the SP2/0-Ag14 myeloma cells were washed several times with serum-free DMEM before fusing them in the presence of 1 mL polyethylene glycol 3350 (50%). The hybridomas were diluted into complete DMEM supplemented with 20% FCS and aminopterin (HAT medium) and then plated out in eight 96-well tissue culture plates that were pre-coated with peritoneal exudate cells as feeder cells. Over the next 10 days, the hybridomas were fed twice with HAT medium, before testing their CSNs by ELISA.

Spleen cells were immortalized by cell fusion using myeloma cell line SP2/0-Ag14 purchased from the German Collection of Microorganisms and Cell Culture (DSMZ GmbH,

Braunschweig). SP2/0-Ag14 was formed by fusing hyperimmune BALB/c mouse spleen cells with the P3X63Ag8 myeloma. Sp2/0-Ag14 cells do not secrete immunoglobulin, are resistant to 8-azaguanine at 20 μg/mL, lack hypoxanthine-guanine phosphoribosyl-transferase (HGPRT) activity and are HAT sensitive (hypoxanthine 10⁻⁴ M, aminopterin 10⁻⁵ M and thymidine 4×10⁻⁵M).

General Culture Method

For tissue culture, only sterile materials and reagents were used. Hybridomas were cultured at 37° C. and 96% relative humidity in sterile-filtered air containing 6% CO₂.

Cells were grown in complete DMEM supplemented with 2-mercaptoethanol, L-glutamine, stable glutamine, HT-supplement and nonessential amino acids at concentrations recommended by the manufacturer. FCS was used at 10%, 15% or 20% depending on the stage of antibody production. Tissue culture supernatants (CSN) contain on average 1 to 20 μg/mL of antibody and it is recommended to experimentally determine the appropriate dilution of the CSN before each user application (possible range from undiluted to 1/5000).

Selection of Hybridomas (First Screening)

An ELISA was developed for the detection of mouse IgG in hybridoma cell culture supernatants. This test is the same as the one described for the detection of mouse IgG in antisera described above.

Cells from wells with positive CSN were transferred to 48-well plates and cultivated for a couple of days. During this time, an ELISA was carried out to confirm the stable production of IgG antibodies with specificity for the hapten A-OVA-conjugate. CSN were also tested against an irrelevant hapten-OVA-conjugate (negative antigen control) in order to exclude non-specific antibodies. The selection phase was kept as short as possible in order to prevent non-specific hybridomas from overgrowing antigen-specific hybridomas.

Selection of Monoclonal, Pan-Reactive Antibodies (Second Screening)

The BIA (Biomolecular Interaction Analysis) assay was used for the selection of monoclonal, pan-reactive antibodies. One flow cell on a CMS sensor chip was immobilized by amine coupling with polyclonal anti-mouse to a level of approx. 16,000 RU. After establishing the concentrations to be used, a method was made to capture anti-hapten monoclonal antibodies with subsequent injections of duocarmycin-containing antibody-drug conjugates depicted ADCs 1 to 18 in FIG. 2. Anti-hapten monoclonal antibodies were injected at a 1:10 dilution (approx. 100 μg/mL) 5 μL/min for 600 s. All duocarmycin-containing antibody-drug conjugates were injected in a 1:500 dilution in HBS-EP (approx. 20 μg/mL) at the same flow rate for 60 s. Dissociation was observed for 300 s. Regeneration with 10 mM glycine HCl a flow rate of 5 μL/min was carried out for 3 minutes after all the duocarmycin-containing antibody-drug conjugates were injected. The method was run in common HBS-EP buffer on flow cells 3 and 4. The data collection rate was 10 Hz and the assay temperature was 25° C.

The concentration of the purified anti-hapten A, B and C clones was estimated with a spectrophotometer at A280 nm. The molar extinction coefficient used was c=1.422.

Cloning of Stable, Antibody-Producing (Hybridoma) Cell Lines

Cells from wells with positive CSN were cloned as soon as possible. Positive monoclonal cell lines were cloned for a second time in order to ensure the monoclonality of the hybridomas. The method of limiting dilution was used for both cloning procedures. After 6-8 days of cell culture, all plates were inspected by light microscopy to identify wells containing single cell colonies. The cells in the identified wells were fed with fresh medium and after 3 more days of culture the CSN were tested by ELISA. Cells from all positive wells were passaged several times in wells of 24-well plates to identify the best clones with regard to cell growth and ELISA signal strength. Selected clones were recloned and bulk-grown for cryopreservation. In particular after the first cloning, clones of four primary cultures continued to produce antibodies against hapten A in case the immunogen of interest used was hapten-carrier protein conjugate A, clones of eight primary cultures continued to produce antibodies against hapten B in case the immunogen of interest used was hapten-carrier protein conjugate B and two primary cultures continued to produce antibodies against hapten C in case the immunogen of interest used was hapten-carrier protein conjugate C.

These primary cultures were cloned for a second time.

Antibody Characterization

Hybridoma cell culture supernatants obtained after the second cloning were tested as follows: a) Determination of the heavy and light chain immunoglobulin isotype using a “Mouse Monoclonal Antibody Isotyping Test Kit” from Serotec; b) Mycoplasma testing and certification by Greiner Bio-One (Frickenhausen, Germany).

Monoclonal, Pan-Reactive Antibodies

Using the methods described above, three monoclonal, pan-reactive antibodies were obtained, i.e., A-15-5-4, B-6-2-10 and C-16-1-7 derived from hapten A, B and C, respectively.

| Antibody | Heavy chain | Light chain |
|---|---|---|
| A-15-5-4 | IgG1 | κ |
| B-6-2-10 | IgG2a | λ |
| C-16-1-7 | IgG2b | κ |

The VH and VL amino acid sequences of said three antibodies are presented herein below as SEQ ID NO:1 and SEQ ID NO:2, SEQ ID NO:3 and SEQ ID NO:4, and SEQ ID NO:5 and SEQ ID NO:6, respectively.

Antibody Application: PK Assay

A coated anti-duocarmycin ELISA was developed to determine the concentration of antibody-drug conjugate (ADC) molecules containing anti-HER2 antibody trastuzumab and duocarmycin as the drug. The main objective for this assay was to determine the concentration of ADC molecules containing the above-mentioned duocarmycin-derived DNA alkylating moiety in PK samples. The assay was designed with an anti-duocarmycin capture step and detection with biotinylated anti-idiotype trastuzumab Fab and streptavidin-HRP. The assay has been tested to be applicable for four different duocarmycin-containing antibody-drug conjugates (ADC-1 to ADC-4 of FIG. 2) in human, mouse, rat and cynomolgus plasma. Twenty-time concentrated duocarmycin-containing antibody-drug conjugate samples in pooled human plasma were aliquoted to be used as calibration curve samples and QC samples. The assay was performed in an anti-duocarmycin pan-reactive antibody (e.g. A-15-5-4, B-6-2-10 or C-16-1-7) coated 96-well plate. Between all incubation steps the assay plate was washed four times with 300 μL PBS-Tween. Subsequent incubations were: sample (biological or non-biological sample with duocarmycin-containing antibody-drug conjugate), biotinylated anti-idiotype trastuzumab Fab, and Streptavidin HRP. The assay range was found to be 7.5 ng/mL-45 ng/mL×dilution factor.

1. A monoclonal antibody that specifically binds antibody-drug conjugates containing a DNA alkylating moiety of formula (I) 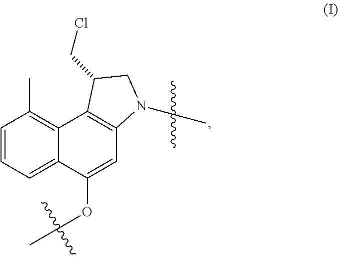 [SMILES: *Oc1cc2c(c3c(C)cccc13)[C@H](CCl)CN2*]
wherein said antibody specifically binds at least two different antibody-drug conjugates that contain said DNA alkylating moiety.

2. The monoclonal antibody according to claim 1 that specifically binds at least three different antibody-drug conjugates that contain said DNA alkylating moiety.

3. The monoclonal antibody according to claim 1 that specifically binds duocarmycin containing ADCs of formula (II) 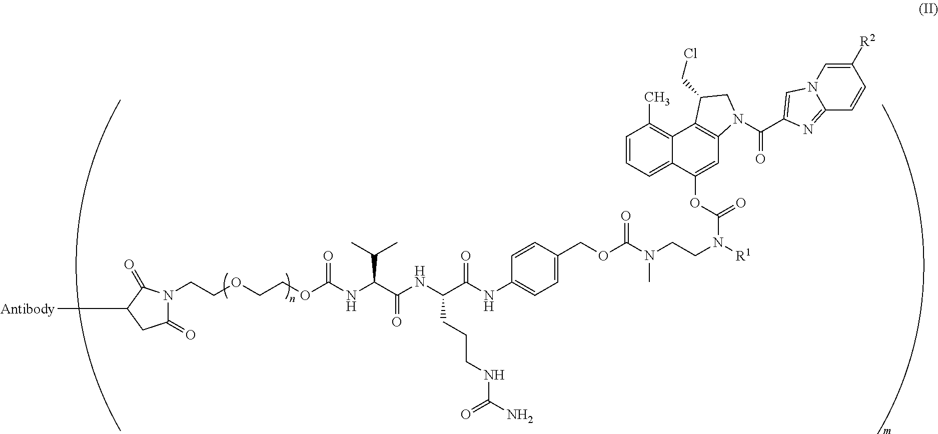
wherein
n is 0-3
m is 1-6 and represents an average drug-to-antibody ratio of from 1 to 6;
R¹ is selected from the group consisting of 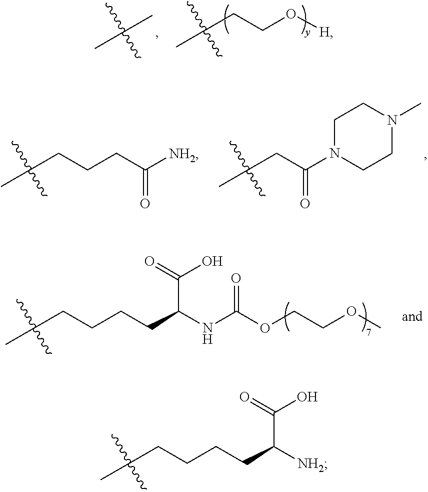
y is 1-16, and
R² is selected from the group consisting of

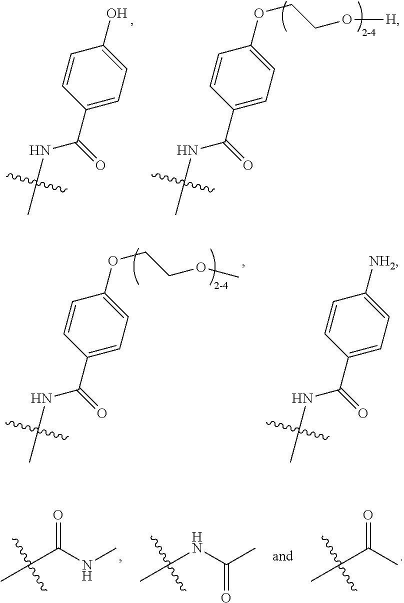

4. The monoclonal antibody according to claim 1 obtainable by raising antibodies against hapten A, B or C 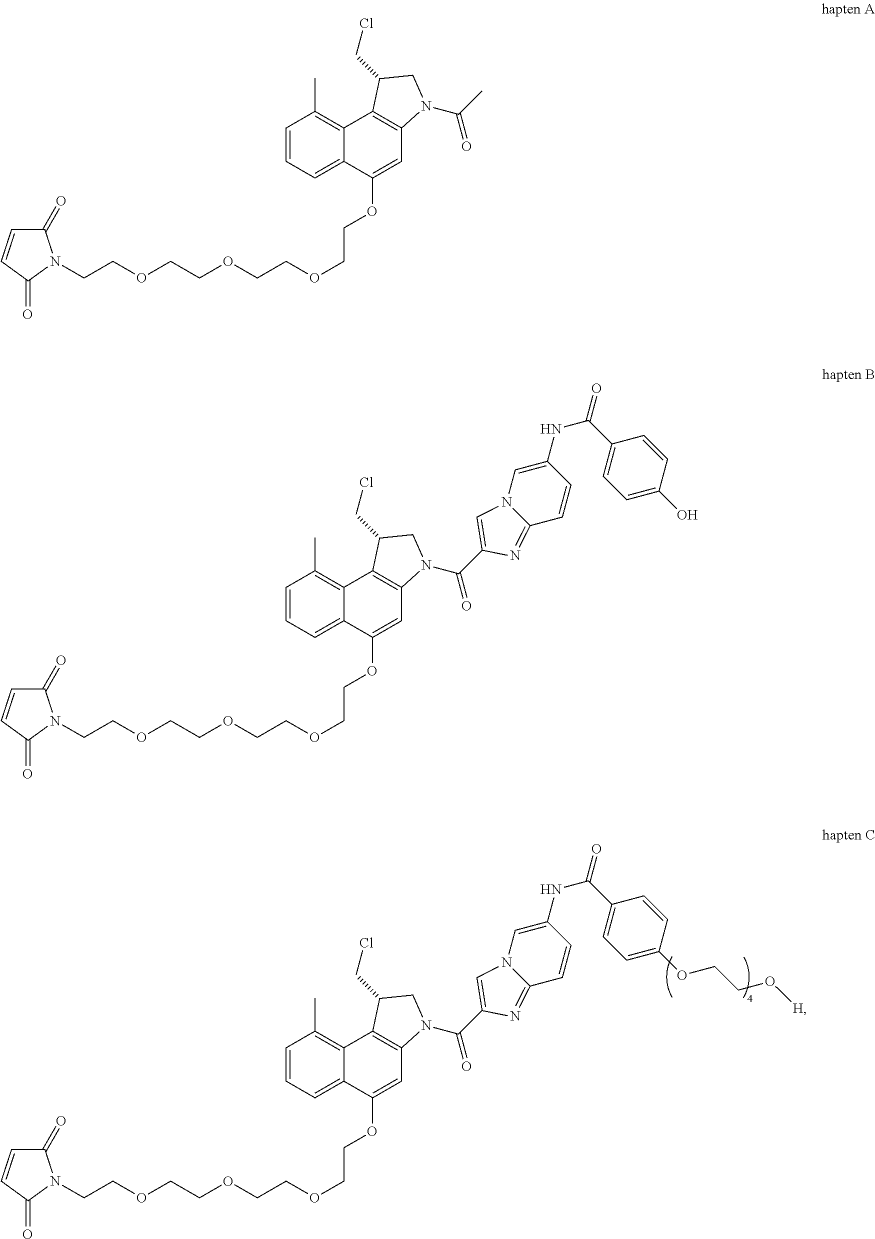
after conjugating the hapten to a carrier protein and immunizing mice with the hapten-carrier protein conjugate.

5. The monoclonal antibody according to claim 1, comprising CDR1, CDR2 and CDR3 of the heavy chain variable domain amino acid sequence of SEQ ID No: 1 and CDR1, CDR2 and CDR3 of the light chain variable domain amino acid sequence of SEQ ID No: 2.

6. The monoclonal antibody according to claim 1, comprising CDR1, CDR2 and CDR3 of the heavy chain variable domain amino acid sequence of SEQ ID No: 3 and CDR1, CDR2 and CDR3 of the light chain variable domain amino acid sequence of SEQ ID No: 4.

7. The monoclonal antibody according to claim 1, comprising CDR1, CDR2 and CDR3 of the heavy chain variable domain amino acid sequence of SEQ ID No: 5 and CDR1, CDR2 and CDR3 of the light chain variable domain amino acid sequence of SEQ ID No: 6.

8. A process for making the monoclonal antibody according to claim 1, comprising:
(i) conjugating hapten A, B or C to a carrier protein;
(ii) immunizing mice with a hapten-carrier protein conjugate of step (i);
(iii) recovering splenocytes or lymphocytes from said immunized mice;
(iv) generating hybridomas;
(v) selecting stable, antibody-producing hybridomas;
(vi) contacting antibodies produced by hybridomas of step (v) with a panel of duocarmycin containing antibody drug conjugates; and
(vii) selecting antibodies that are reactive against at least two of the panel of duocarmycin-containing antibody-drug conjugates.

9. A method for detecting, isolating or quantifying a duocarmycin-containing antibody-drug conjugate in a biological or non-biological sample comprising:
(i) subjecting said sample to an immunoassay using the monoclonal antibody according to claim 1; and
(ii) detecting, isolating or quantifying the resulting immune complex.

10. The method according to claim 9, wherein said immunoassay comprises:
(i) providing a solid phase having said monoclonal, pan-reactive antibody bound thereto;
(ii) contacting said solid phase with said biological or non-biological sample;
(iii) washing said support;
(iv) detecting a duocarmycin-containing antibody-drug conjugate attached to said support; and
(v) determining the quantity of said antibody-drug conjugate in said sample.

11. (canceled)

12. A kit for the detection or quantification of a duocarmycin-containing antibody-drug conjugate in a biological or non-biological sample comprising the monoclonal antibody according to claim 1 and instructions for use.

13. The monoclonal antibody according to claim 2 that specifically binds at least five different antibody-drug conjugates that contain said DNA alkylating moiety.

14. The monoclonal antibody according to claim 13 that specifically binds at least ten different antibody-drug conjugates that contain said DNA alkylating moiety.

15. The monoclonal antibody according to claim 14 that specifically binds at least fifteen different antibody-drug conjugates that contain said DNA alkylating moiety.

16. The monoclonal antibody according to claim 3, wherein n is 0 or 1 in the duocarmycin containing ADCs of formula (II).

17. The monoclonal antibody according to claim 3, wherein m is 1-4, and represents an average drug-to-antibody ratio of from 1 to 4, in the duocarmycin containing ADCs of formula (II).


SEQUENCE LISTING (6 sequences)

SEQ ID NO: 1 - protein, 122 aa, Mus musculus
QVQLQQPGAELVKPGASVKLPCKASGYTFISYYMYWVKQRPGQGLEWIGVINPSNGGTTFNEKFKNKATLTVDKSSSTAYLQLSSLTSEDSAVYYCTCLNYYGSSYWYFDVWGAGTTVTVSS

SEQ ID NO: 2 - protein, 112 aa, Mus musculus
DVVMTQTPLSLPVSLGDQASIFCRSSQSLVHSDGNTYLHWYLQKPGQSPKLLIYTVSNRFSGVPDRFSGSGSGTDFTLKISRVEAEDLGVYFCSQSTHVPWTFGGGTTLEIK

SEQ ID NO: 3 - protein, 122 aa, Mus musculus
DVKLVESGGGLVKPGGSLKLSCAASGFTFRTYTMSWVRQTPEKRLEWVATISSGGSYTYYPDSMKGRFTISRDNAKNTLYLQMSSLKSEDTAMYYCTRGPFYDVYYFLFAYWGQGTLVTVSA

SEQ ID NO: 4 - protein, 109 aa, Mus musculus
QAVVTQESALTTSPGETVTLTCRSSIGAVTTTNYANWVQEKPDHLFTGLIGGTNNRAPGVPARFSGSLIGDKAALTITGAQTEDEAIYFCALWYSNHWVFGGGTKLTVL

SEQ ID NO: 5 - protein, 115 aa, Mus musculus
EVKLVESGGGLVKPGGSLKVSCAASGFTFSSYAMSWVRQIPDKRLEWVASISTGGITYYLDGVKGRFTISRDNARNILYLQMSSLRSEDTAIYYCARWSYAMDYWGQGTSVTVSS

SEQ ID NO: 6 - protein, 108 aa, Mus musculus
QIVLTQSPAIMSASLGERVTMTCTASSSVSSSYLHWYQQKPGSSPKVWIYSTSNLASGVPARFRGSGSGTSYSLTISSMEAEDAATYYCHQYHRSPPTFGGGTKLEIK